(19) United States
(12) Patent Application Publication
(10) Pub. No.: US 2013/0204151 A1
(43) Pub. Date: Aug. 8, 2013

(54) Augmented Incentive Spirometer

(71) Applicants: Sebo Amirkhanian, Tujunga, CA (US); Moises Carpio, Fullerton, CA (US)

(72) Inventors: Sebo Amirkhanian, Tujunga, CA (US); Moises Carpio, Fullerton, CA (US)

(21) Appl. No.: 13/367,452

(22) Filed: Feb. 7, 2012

(51) Int. Cl. A61B 5/087

(52) U.S. Cl. 600/538

(57) ABSTRACT

A novel augmented incentive spirometer that will deliver pressurized oxygen, or air when the patient inhales, therefore increasing the inspired volume over the amount of volume generated only by the patient's effort.

### CROSS-REFERENCE TO RELATED APPLICATIONS

Not Applicable

### STATEMENT REGARDING FEDERALLY SPONSORED RESEARCH OR DEVELOPMENT

Not Applicable

### INCORPORATION-BY-REFERENCE OF MATERIAL SUBMITTED ON A COMPACT DISC

Not Applicable

### BACKGROUND OF THE INVENTION

1. Field of the Invention

The present invention relates generally to respiratory therapy devices of the character used in daily medical practice to prevent pulmonary complications in patients who have had chest or abdominal operations. More particularly, the invention concerns a novel augmented incentive spirometer that will deliver pressurized oxygen, or air when the patient inhales, therefore increasing the inspired volume over the amount of volume generated only by the patient's effort.

A spirometer is typically defined as an apparatus for measuring the volume of air inspired and expired by the lungs. More particularly, the spirometer is a differential pressure transducer for the measurements of respiration flow rates. The spirometer records the amount of air and the rate of air that is breathed in and out over a specified period of time. The commonly used prior art devices are simple, single use, disposable devices that are relatively inexpensive. The prior art spirometers are designed to encourage patients to take deep breaths that can be quantifiably and objectively seen as the patient's inspiration creates flow and a measured volume of air or air-mix flows into the lungs. The volume of air depends on the effort of the patient when taking deep breaths and the higher the volume generated by the patient, the greater the benefit. This increase in the inspired volume leads to the prevention of lung atelectasis or airways collapse.

For the more compromised, weaker, and seriously ill patients, the prior art incentive spirometers simply will not provide the desired results. Accordingly, the thrust of the present invention is to provide an augmented incentive spirometer of unique design that will deliver pressurized air when the patient inhales, therefore increasing the inspired volume over the amount of volume generated only by the patient's effort.

2. Description of Related Art Including Information Disclosed Under 37 CFR 1.97 and 1.98

A quite popular prior art incentive spirometer is commercially available under the designation Coach 2 from the DHD Healthcare Corporation of Wampsville, N.Y. The Coach 2 incentive spirometer, which is available is available in 4,000-mL and 2,500-mL capacities, includes a one-way valve, universal graphics, a brightly colored piston, and a bedrail holder.

Another prior art spirometer is disclosed in U.S. Pat. No. 3,395,699 issued to Beasley. The Beasley spirometer comprises a case, an expandable-contractible bellows supported on the case and having an inlet and an outlet. The bellows is arranged to receive gas through the inlet and expel it through the outlet during movement between expanded and contracted conditions. A first valve means is provided in the inlet to permit flow to the bellows but functions to block flow in the opposite direction. A second valve means is provided for closing the outlet. The second valve means is movable to an open position to permit gas to be expelled from the bellows. Also provided is a pneumatic actuating means that is responsive to a pressure signal from moving the second valve means to its open position and means responsive to the bellows moving to its contracted condition for causing the second valve means to move to its closed position.

U.S. Pat. No. 5,107,830 issued to Younes concerns a lung ventilator device in which ventilation to a patient is provided in response to patient effort. The free flow of gas from a piston, or similar air source, in response to patient inhalation is detected, the instantaneous rate and volume of flow are measured, and the measurements are used as control signals to a drive motor for the piston to move the piston to generate a pressure which is proportional to the sum of measured and suitably amplified rate and volume of flow signals. Since the command signal to the pressure generator only changes subsequent to, and not in advance of, a change in flow and volume, the ventilator is subservient to the patient and provides a proportional assist to patient ongoing breathing effort during inspiration.

### BRIEF SUMMARY OF THE INVENTION

By way of brief summary, one form of the augmented incentive spirometer of the present invention comprises a housing having first and second portions, the first portion having an outlet and the second portion including a reservoir having an inlet and an outlet. Carried within the first portion of the housing is a piston that is movable between a first position and a second position. Connected to the housing is a combination cylinder that has a first inlet in communication with the reservoir of the second portion of the housing, a second inlet in communication with the first portion of said housing and a first outlet in communication with the outlet of the second portion of the housing. Connected to the combination cylinder and in communication with the first portion of the housing is a patient inhalation subassembly. A source of oxygen is connected to the second portion of said housing for controllably introducing oxygen into the reservoir of the second portion via a control valve that controls the flow of oxygen into the reservoir. As the piston moves between its first and second positions, an operating member which interconnects the piston with the control valve controllably operates the control valve to regulate the flow of oxygen into the reservoir.

With the foregoing in mind, it is an object of the present invention to provide a novel augmented incentive spirometer that will deliver pressurized oxygen to the patient when the patient inhales, therefore increasing the inspired volume over the amount of volume generated only by the patient's effort.

Another object of the invention is to provide to provide an apparatus of the character described which in operation is not totally dependent upon the patient's cooperation.

Another object of the invention is to provide to provide an apparatus of the class described that will provide an augmented tidal volume during the patient inspiration step, even if the patient is unable to generate sufficient negative inspiratory pressure to generate adequate tidal volume.

Another object of the invention is to provide an apparatus of the character described in the preceding paragraphs that will enable even debilitated patients to generate a negative inspiratory pressure capable of triggering the system. Accordingly, in using the apparatus of the invention the patient does not need keep inhaling to create a tidal volume that is sufficient to open up small airways and thereby prevent athelectasis. Additionally, once the patient inhales, the pressurized oxygen-air flows into the patient's lungs and becomes independent of the negative inspiratory pressure generated by the patient.

Another object of the invention is to provide to provide an apparatus of the aforementioned character that includes a one-way safety valve that is disposed within an outlet port formed in the combination cylinder.

Another object of the invention is to provide to provide an apparatus of the character described in which the flow of the air oxygen mixture to the patient is directly proportional to the negative inspiratory pressure (NIP) created by the patient during the inhalation step.

Another object of the invention is to provide an augmented incentive spirometer of the class described that is easy to use with a minimum amount of instruction.

Still another object of the invention is to provide an augmented incentive spirometer that is of a simple, inexpensive construction and one which is highly reliable in operation.

The foregoing objectives, as well as other features and advantages of the present invention, will become readily apparent from the following detailed description and the accompanying drawings.

### BRIEF DESCRIPTION OF SEVERAL VIEWS OF THE DRAWINGS

FIG. 1 is a generally perspective view of one form of the augmented incentive spirometer of the present invention.

FIG. 2 is a cross-sectional view of the augmented incentive spirometer shown in FIG. 1.

### DETAILED DESCRIPTION OF THE INVENTION

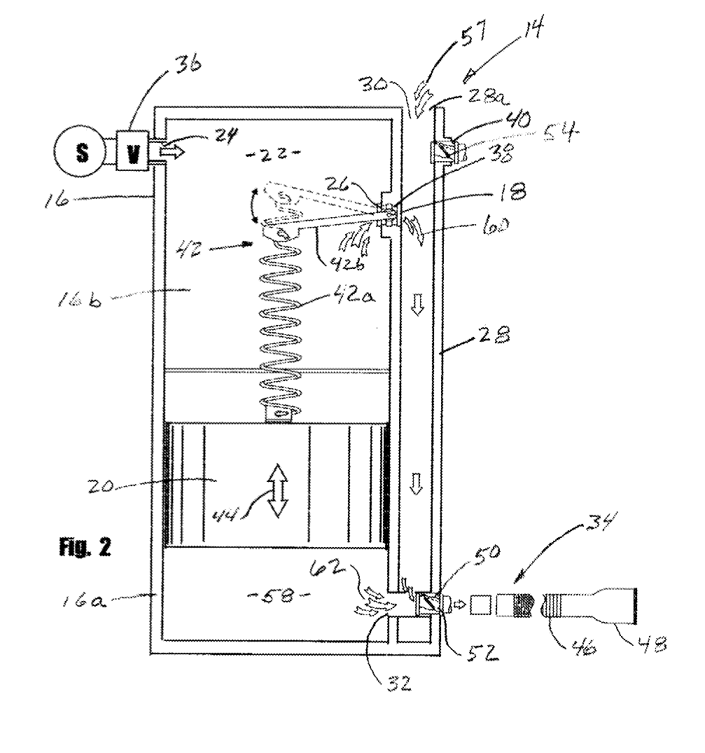

Referring to the drawings, one form of the augmented incentive spirometer of the invention is there shown and generally designated by the numeral 14. Augmented incentive spirometer 14 here comprises a housing 16 having first and second portions 16a and 16b. First portion 16a, which is generally cylindrical in shape, has an outlet 18. Mounted within first portion 16a for movement there within between a first position and a second position is a generally cylindrical piston 20. As best seen in FIG. 2, second portion 16b includes a reservoir 22 having an inlet 24 and an outlet 26.

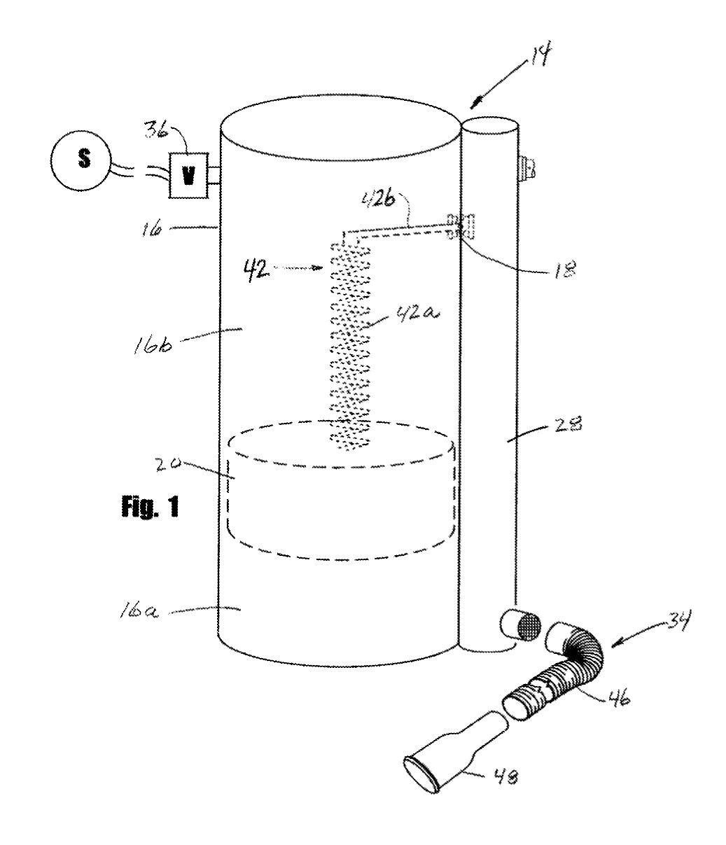

Connected to housing 16 is an elongate, generally cylindrically shaped combination cylinder 28 that has an open end 28a that defines a first inlet 30. Combination cylinder 28 also has a second inlet 32 that is in communication with the first portion 16a of said housing 16 in the manner illustrated in FIG. 2 of the drawings. Connected to combination cylinder 28 is a patient inhalation subassembly 34, the details of construction of which will presently be described. (See also FIG. 1.)

Connected to second portion 16b of housing 16 is a source of oxygen “S” that communicates with reservoir 22 via a conventional pressure breaking valve 36 that is connected to second portion 16b proximate inlet the 24. Pressure breaking valve 36 maintains the flow of oxygen from the source of oxygen into reservoir 22 at a pressure of between about 45 and about 50 pounds per square inch (psi) and maintains the rate of flow of oxygen into the reservoir at between about 600 and about 800 liters per minute.

Also connected to second portion 16b of housing 16 proximate the outlet 26 is a control valve 38 which, in a manner presently to be described, functions to control the flow of oxygen from reservoir 22 into combination cylinder 28. For a purpose presently to be described, combination cylinder 28 is provided with a first outlet 40. Operably associated with control valve 38 is a control valve operating assembly 42 that functions to operate the control valve as piston 20 moves within the first portion of the housing in the manner indicated by the arrow 44 of FIG. 2 between the first and second positions. In the present form of the invention, operating assembly 42 comprises a coil spring 42a and a valve operating member 42a that is connected to control valve 38. As best seen in FIG. 2 of the drawings, coil spring 42a interconnects the distal end of the valve operating member with the piston 20.

In the present form of the invention, the patient inhalation subassembly 34 comprises a flexible tube 46 and a patient mouthpiece 48 that is connected to the flexible tube. As illustrated in FIG. 2, flexible tube 46 is connected to a one-way safety valve 50 that is connected to combination cylinder 28 proximate a second outlet 52. For further operational safety, a safety valve 54 is carried by the combination cylinder 28 proximate first outlet 40. In the present form of the invention, safety valve 54 opens if the pressure in the combination cylinder 28 becomes greater than a pre-set pressure, which in this case is about 6 pounds psi. In using the augmented incentive spirometer of the invention, the source of oxygen “S” is first connected to the pressure breaking valve 36 in the manner shown in FIG. 2 and oxygen is permitted to flow into reservoir 22 at a prescribed pressure and at a prescribed rate of flow. This done, the patient is requested to take a deep breath using the mouthpiece 48 of the inhalation assembly 34. In the manner indicated by the arrows 57 in FIG. 2, this step causes room air to enter the open end 28a of combination cylinder 28 that defines a first inlet 30. Inhalation by the patient also creates a negative pressure in the chamber 58 of the lower portion of the housing. This negative pressure causes the piston 20 to move downwardly within chamber 58, which, in turn, causes the operating member 42b to pivot downwardly against the urging of spring 42a in a manner to open control valve 38. Uniquely, the extent to which the control valve is opened by the operating member is directly proportional to the negative inspiratory pressure (NIP) created by the patient during the inhalation step. As the control valve opens, oxygen will flow into the combination cylinder in the manner illustrated by the arrows 60 in FIG. 2 where it mixes with the room air. As indicated by the arrows 62 in FIG. 2, as the patient continues to inhale the mixture of room air and oxygen will flow into the patient's mouth simulating the “French kiss” positive pressure breathing described in the literature. Uniquely, the pressurized air oxygen mixture will continue to flow to the patient even when the patient is no longer inhaling. Accordingly, the patient does not need to continue inhaling to receive the appropriate volume of air oxygen mixture.

At the point at which the negative inspiratory pressure, which is created by the patient during the inhalation step, is lower than the pressurized air oxygen mixture at the one-way safety valve 50, the piston 20 will return to its original position. As the piston returns to its original position, the control valve 38 will close, thereby preventing further flow of the pressurized air oxygen mixture to the patient.

Having now described the invention in detail in accordance with the requirements of the patent statutes, those skilled in this art will have no difficulty in making changes and modifications in the individual parts or their relative assembly in order to meet specific requirements or conditions. Such changes and modifications may be made without departing from the scope and spirit of the invention, as set forth in the following claims.

1. An augmented incentive spirometer comprising:
(a) a housing having first and second portions, said first portion having an outlet and said second portion including a reservoir having an inlet and an outlet;
(b) a piston carried by said first portion of said housing for movement there within between a first position and a second position;
(c) a combination cylinder connected to said housing, said combination cylinder having:
(i) a first inlet;
(ii) a second inlet in communication with said first portion of said housing; and
(iii) a first outlet in communication with said outlet of said second portion of said housing;
(d) a patient inhalation subassembly connected to said combination cylinder and being in communication with said first portion of said housing;
(e) a source of oxygen connected to said second portion of said housing, said source of oxygen being in communication with said inlet of said second portion of said housing;
(f) a control valve carried by said second portion of said housing for controlling fluid flow through said outlet of said second portion of said housing; and
(g) a control valve operating assembly operably associated with said control valve for operating said control valve as said piston moves within said first portion of said housing between said first position and said second positions.

2. The augmented incentive spirometer as defined in claim 1 in which said operating assembly comprises a valve operating member and a coil spring inter-connecting said piston with said valve operating member.

3. The augmented incentive spirometer as defined in claim 1 in which said combination cylinder includes a second outlet and in which said augmented incentive spirometer further includes a one-way safety valve carried by said second portion of said housing for controlling fluid flow through said second outlet of said second portion of said housing.

4. The augmented incentive spirometer as defined in claim 1, wherein said control valve operating member comprises a spring.

5. The augmented incentive spirometer as defined in claim 1 in which said patient inhalation subassembly comprises a flexible tube and a patient mouthpiece connected to said flexible tube.

6. The augmented incentive spirometer as defined in claim 1, further comprising a pressure breaking valve carried by said second portion of said housing for controlling the flow of oxygen into said reservoir of second portion of said housing.

7. The augmented incentive spirometer as defined in claim 5 in which said pressure breaking valve maintains the flow of oxygen from said source of oxygen into said reservoir of second portion of said housing at a pressure of between about 45-50 pounds per square inch and maintains the rate of flow of oxygen into said reservoir of second portion of said housing at between about 600 and 800 liters per minute.

8. An augmented incentive spirometer comprising:
(a) A housing having first and second portions, said first portion having an outlet and said second portion including a reservoir having an inlet and an outlet;
(b) A piston carried by said first portion of said housing for movement there within between a first position and a second position;
(c) a combination cylinder connected to said housing, said combination cylinder having:
(i) a first inlet;
(ii) a second inlet in communication with said first portion of said housing;
(iii) a first outlet in communication with said outlet of said second portion of said housing; and
(iv) a second outlet;
(d) a patient inhalation subassembly connected to said combination cylinder and being in communication with said first portion of said housing;
(e) a source of oxygen connected to said second portion of said housing, said source of oxygen being in communication with said inlet of said second portion of said housing;
(f) a control valve carried by said second portion of said housing for controlling fluid flow through said outlet of said second portion of said housing; and
(g) a control valve operating assembly operably associated with said control valve for operating said control valve as said piston moves within said first portion of said housing between said first position and said second positions, said control valve operating assembly comprising a valve operating member and a coil spring interconnecting said piston with said valve operating member.

9. The augmented incentive spirometer as defined in claim 8 further including a one-way safety valve carried by said second portion of said housing for controlling fluid flow through said second outlet of said second portion of said housing.

10. The augmented incentive spirometer as defined in claim 8 in which said patient inhalation subassembly comprises a flexible tube and a patient mouthpiece connected to said flexible tube.

11. The augmented incentive spirometer as defined in claim 8, further comprising a pressure breaking valve carried by said second portion of said housing for controlling the flow of oxygen into said reservoir of second portion of said housing.

12. The augmented incentive spirometer as defined in claim 11 in which said pressure breaking valve maintains the flow of oxygen from said source of oxygen into said reservoir of second portion of said housing at a pressure of between about 45-50 pounds per square inch and maintains the rate of flow of oxygen into said reservoir of second portion of said housing at between about 600 and 800 liters per minute.

13. An augmented incentive spirometer comprising:
(a) a housing having first and second portions, said first portion having an outlet and said second portion including a reservoir having an inlet and an outlet;
(b) a piston carried by said first portion of said housing for movement there within between a first position and a second position;
(c) a combination cylinder connected to said housing, said combination cylinder having:
(i) a first inlet;
(ii) a second inlet in communication with said first portion of said housing;
(iii) a first outlet in communication with said outlet of said second portion of said housing; and
(iv) a second outlet;
(d) a patient inhalation subassembly connected to said combination cylinder and being in communication with said first portion of said housing, said patient inhalation subassembly permitting inhalation by the patient at a controlled rate and comprising a flexible tube and a patient mouthpiece connected to said flexible tube;
(e) a source of oxygen connected to said second portion of said housing, said source of oxygen being in communication with said inlet of said second portion of said housing;
(f) a pressure breaking valve carried by said second portion of said housing for controlling the flow of oxygen into said reservoir of second portion of said housing;
(g) a control valve carried by said second portion of said housing for controlling fluid flow through said outlet of said second portion of said housing at a rate proportional to the rate of inhalation by the patient; and
(h) a control valve operating assembly operably associated with said control valve for operating said control valve as said piston moves within said first portion of said housing between said first position and said second positions, said control valve operating assembly comprising a valve operating member and a coil spring interconnecting said piston with said valve operating member.

14. The augmented incentive spirometer as defined in claim 13 further including a one-way safety valve carried by said second portion of said housing for controlling fluid flow through said second outlet of said second portion of said housing.

15. The augmented incentive spirometer as defined in claim 11 in which said pressure breaking valve maintains the flow of oxygen from said source of oxygen into said reservoir of second portion of said housing at a predetermined pressure and maintains the rate of flow of oxygen into said reservoir of second portion of said housing at a predetermined rate.

16. The augmented incentive spirometer as defined in claim 15 in which said pressure breaking valve maintains the flow of oxygen from said source of oxygen into said reservoir of second portion of said housing at a predetermined pressure of between about 45-50 pounds per square inch and maintains the rate of flow of oxygen into said reservoir of second portion of said housing at a predetermined rate of between about 600 and 800 liters per minute.

17. An augmented incentive spirometer comprising:
(a) a generally cylindrically shaped housing having first and second portions, said first portion having a reservoir having an outlet and said second portion including a reservoir having an inlet and an outlet;
(b) a cylindrically shaped piston carried by said first portion of said housing for movement there within between a first position and a second position;
(c) a combination cylinder connected to said housing, said combination cylinder having:
(i) a first inlet;
(ii) a second inlet in communication with said first portion of said housing;
(iii) a first outlet in communication with said outlet of said second portion of said housing; and
(iv) a second outlet;
(d) a patient inhalation subassembly connected to said combination cylinder and being in communication with said first portion of said housing, said patient inhalation subassembly permitting inhalation at a controlled rate and comprising a flexible tube and a patient mouthpiece connected to said flexible tube;
(e) a source of oxygen connected to said second portion of said housing, said source of oxygen being in communication with said inlet of said second portion of said housing;
(f) a pressure breaking valve carried by said second portion of said housing for controlling the flow of oxygen into said reservoir of second portion of said housing;
(g) a control valve carried by said second portion of said housing for controlling fluid flow through said outlet of said second portion of said housing at a rate proportional to the rate of inhalation by the patient; and
(h) a control valve operating assembly operably associated with said control valve for operating said control valve as said piston moves within said first portion of said housing between said first position and said second positions. said control valve operating assembly comprises a valve operating member and a coil spring interconnecting said piston with said valve operating member.

18. The augmented incentive spirometer as defined in claim 17 further including a one-way safety valve carried by said second portion of said housing for controlling fluid flow through said second outlet of said second portion of said housing.

19. The augmented incentive spirometer as defined in claim 17 in which said pressure breaking valve maintains the flow of oxygen from said source of oxygen into said reservoir of second portion of said housing at a predetermined pressure and maintains the rate of flow of oxygen into said reservoir of second portion of said housing at a predetermined rate.

20. The augmented incentive spirometer as defined in claim 19 in which said pressure breaking valve maintains the flow of oxygen from said source of oxygen into said reservoir of second portion of said housing at a predetermined pressure of between about 45-50 pounds per square inch and maintains the rate of flow of oxygen into said reservoir of second portion of said housing at a predetermined rate of between about 600 and 800 liters per minute.
